(19) United States
(12) Patent Application Publication
(10) Pub. No.: US 2025/0018168 A1
(43) Pub. Date: Jan. 16, 2025

(54) DEVICE AND METHOD FOR MONITORING AND OPTIMIZING A TEMPORAL TRIGGER STABILITY

(71) Applicant: Xenios AG, Heilbronn (DE)

(72) Inventors: Matthias Heinke, Rudolstadt (DE); Johannes Hörth, Offenburg (DE); Wofgang Schultz, Freiburg (DE)

(21) Appl. No.: 18/712,957

(22) PCT Filed: Nov. 24, 2022
(86) PCT No.: PCT/EP2022/083192

§ 371 Date: May 23, 2024

(30) Foreign Application Priority Data
Nov. 24, 2021 (DE) 10 2021 005 828.3

(51) Int. Cl. A61M 60/117; A61B 5/352; A61M 60/515

(52) U.S. Cl. CPC A61M 60/117; A61B 5/352; A61M 60/515

(57) ABSTRACT

The present invention relates to devices for monitoring and optimizing a temporal trigger stability of an extracorporeal circulatory support and to control and regulation units for extracorporeal circulatory support comprising such a device and corresponding methods. Accordingly, a device (10) for monitoring a temporal trigger stability of an extracorporeal circulatory support is suggested, which is adapted to receive a first data set (14) of a measurement of an ECG signal of a supported patient over a predefined period of time. The device (10) comprises an evaluation unit (16) adapted to determine or identify a plurality of R-triggers (26) from the first data set (14), wherein the evaluation unit (16) is furthermore adapted to receive or provide a second data set (20) comprising evaluated ECG signals and a plurality of R-triggers (28) and to selectively map the second data set (20) onto the first data set (14). The device is furthermore adapted to output a signal (22) indicative of a temporal distance of successive R-triggers (26) from the first data set (14) and successive R-triggers (28) from the second data set (20) being mapped thereon.

### TECHNICAL FIELD

The present invention relates to devices for monitoring and optimizing a temporal trigger stability of an extracorporeal circulatory support as well as control and regulation units for extracorporeal circulatory support comprising such a device and corresponding methods.

### TECHNOLOGICAL BACKGROUND

In order to stabilize a patient's condition in the event of a failure of pumping performance or pumping function of the heart extracorporeal circulatory support systems have been developed, which provide mechanical support and can be rapidly connected to the circulatory system. They can improve the blood flow and perfusion of organs, including the coronary vessels of the heart and avoid a hypoxic state. For example, a blood pump may be connected to a venous access via a venous cannula and an arterial access via an arterial cannula to aspirate or suck and feed blood, respectively, to provide blood flow from a side having a low pressure, such as via an oxygenator, to a side having a higher pressure, thereby supporting the patient's circulation.

Measurement signals from an electrocardiogram (ECG) may be detected and used to control the extracorporeal support, whereby corresponding characteristic amplitudes may be determined for different cardiac cycle phases. For example, an R-spike or R-wave characteristic of the systolic phase of the cardiac cycle is usually readily distinguishable from other phases of the cardiac cycle. The R-wave can thus serve as an R-trigger with a predetermined latency to control a blood pump in a successive diastolic phase. In the publication DE 10 2020 004 698 A1 the temporal and/or spatial determining of the amplitude, e.g. the R-Spike, is taught for optimizing the signal amplitudes. In this manner, noise signals may be taken into account in the respective cardiac cycles.

Different ECG leads may be provided for providing an ECG signal, e.g., transthoracic, transesophageal, or intracardiac leads, which are positioned at or inserted into different anatomical regions. This causes some variability in the measurement signal. Furthermore, stimulation-related, pathophysiological, or artifact-related disturbances can cause significant fluctuations or noise signals in the ECG signal that make it difficult to determine the amplitudes or ventricular signal shapes in the cardiac cycle, such that the desired amplitude or signal shape or waveform may not be detected or determined.

This not only results in inconsistency with respect to monitoring and optimizing cardiac action and performance. Rather, a control of the extracorporeal circulatory support, which uses the amplitude and/or signal shape as a trigger signal, may be activated at the wrong time, such that support does not take place in the intended cardiac cycle phase. Furthermore, this provides a reduced trigger stability. For example, if an R-wave is to serve as a basis for a trigger signal, time intervals between two successive R-trigger signals may vary, if the amplitudes and/or signal shapes corresponding thereto cannot be correctly detected. Such an R-trigger instability leads to an asynchronous control of extracorporeal circulatory support, which is unfavorable for the supported patient and hemodynamically suboptimal.

It is also problematic that a potentially existing or occurring R-trigger instability is not immediately identifiable and had to be checked in practice using manual procedures by medically trained personnel. This requires a complex and time-consuming analysis and does not enable an online correction or correction of a currently occurring R-trigger instability in a supported patient.

Accordingly, there is a need to improve the verification of the temporal trigger stability of an extracorporeal circulatory support, preferably under different physiological and/or clinical conditions, and to optimize the temporal electrical trigger stability during circulatory support.

### SUMMARY OF THE INVENTION

Based on the known state of the art, it is an object of the present invention to provide an improved monitoring and optimization of the temporal trigger stability of an extracorporeal circulatory support.

This object is achieved by the independent claims. Preferred embodiments are defined by the dependent claims, the description, and the Figures.

Accordingly, a device for monitoring and optimizing a temporal trigger stability of an extracorporeal circulatory support is suggested, which is adapted to receive a first data set of a measurement of an ECG signal of a supported patient over a predefined period of time, wherein the device comprises an evaluation unit which is adapted to determine or identify a plurality of R-triggers from the first data set. The evaluation unit is furthermore adapted to receive or provide a second data set comprising evaluated ECG signals and a plurality of R-triggers and to selectively map the second data set onto the first data set. Furthermore, the device is adapted to output a signal indicative of a temporal distance of successive R-triggers from the first data set and successive R-triggers from the second data set mapped thereon. Typically, the annotation of the R-trigger in the second data set is performed by communicating corresponding data. However, the R-trigger can also be determined exactly by differential analysis (in the leads).

The core of the invention hence lies in particular in the determining of R-R intervals for at least two data sets that are retrieved from ECG-signals as well as their subsequent mapping to each other. The at least two data sets may be generated independently from each other by at least two independent ECG-signal recordings or by a single ECG-signal recording, forming the basis for the generation of at least two data sets. The determining of at least two R-R intervals requires the detection or recording of at least three R-spikes, if directly subsequent cardiac cycles with their corresponding R-R intervals are taken into account (R₁, R₂, R₃). This enables the determining of a first R-R interval, i.e. a time duration or temporal distance between two subsequent R-spikes (R₁-R₂=R_{interval 1}), and a second R-R interval, i.e. the time duration of another sequence of R-spikes, e.g. R₂-R₃=R_{interval 2}). However, if both of the intervals that are to be determined are not directly subsequent to each other (e.g. if they are separated by one or two cardiac cycles), the determining of a first R-R interval (determined by two subsequent R-spikes) and a second R-R interval (determined by two other subsequent R-spikes) requires the recording of at least four R-spikes. For example, in case of the intervals R₁-R₂ (first interval) and R₃-R₄ (second interval) or, in case of not considering R₃: R₁, R₂, (R₃), R₄, R₅: R₁-R₂ (first interval) and R₄-R₅ (second interval). In such case the duration between two subsequent R-spikes is determined for two subsequent cardiac cycles, respectively, wherein the determined R-spikes are different. The recording of at least four R-spikes is accordingly also required in case of data sets that are received from independent ECG-signal recordings. Preferably, according to the invention, it is hence provided that exclusively the determining of R-spikes for two subsequent cardiac cycles and the determining of their interval durations are taken into account. The further signals of the ECG are hence preferably not taken into account or may preferably be exclusively used to more specifically determine the R-R intervals.

In this manner, a trigger stability may be automatically detected on the basis of the temporal distance or time interval and, if necessary, adjusted or optimized, for example, on the basis of a corresponding adjustment of the control of the circulatory support. This is because any variability of the ECG signal and an associated change in the R-trigger may lead to a change in the time interval between two successive R-triggers, a so-called R-R interval. However, by means of the mapped second data set, such ECG signals are also sufficiently taken into account. Accordingly, the decisive factor is rather whether the temporal placement of the R-trigger according to the first data set in the respective cardiac cycle corresponds to a placement of the R-trigger according to the second data set and whether the resulting R-R intervals differ from each other. A possibly incorrectly placed or unplaced R-trigger signal may lead to a deviation and a user can be made aware of this circumstance by the signal to be output. On the other hand, a correct placement of the R-trigger with a possibly changed R-R interval does not necessarily lead to an increased trigger instability.

Hence, according to the invention, individual or also recurring anomalies may be detected and R-triggers may be checked or validated on the basis of the second data set. Preferably, corresponding parameters are considered between the first and the second data set. The selective mapping of the second data set may be temporally adapted to the respective cardiac cycle or a corresponding cardiac cycle phase, or may include one or more specific ECG signals that match one or more parameters from the measured ECG signal of the first data set. Thus, a best-fit ECG signal from the second data set may be mapped onto the first data set. However, it is also possible for the second data set to be based on ECG measurements with ECG leads that are at least partially or completely different from the ECG leads of a patient currently provided with circulatory support. For example, the second data set may include transthoracic ECG measurements while the current circulatory support is performed with a left cardiac and/or transesophageal ECG lead. Optimization of R-trigger instability may still be performed, if it can be assumed that the a potentially detected temporal difference is not significantly different for the different ECG leads.

Preferably, the selective mapping comprises temporal and spectral mapping. The combination of a temporal and spectral evaluation or consideration of these factors in the mapping thus enables a spectro-temporal mapping. The weighting of the spectral and temporal components can be variable and, in particular, be advantageously adapted to the available ECG data and/or individual ECG parameters of the first data set.

The first data set may be received by means of an interface of the device, wherein the measured ECG signal may be processed using a memory and/or stored in a storage medium. For example, the ECG signal may be communicatively connected to the evaluation unit via the interface. The evaluation unit may comprise both a memory and a storage medium. Furthermore, the second data set may be received via the interface or multiple ECG signals may be received. Thus, advantageously, the evaluation unit may be adapted to evaluate the ECG signals to provide the second data set.

Preferably, the device is designed as part of an ECG device. However, it may also be communicatively connectable to at least one ECG device via one or the interface(s) described above.

The second data set may comprise a plurality of validated clinical ECG signal measurements and/or simulated ECG signal data. For example, the ECG signals of the second data set may comprise different bradycardic and tachycardie cardiac arrhythmias, which allow for improved and/or more specific mapping, even in case of increased variability of the ECG signal of the first data set.

In order to be able to better verify a trigger stability in case of artifacts, for example, due to the occurrence of stimulation-related interfering signals, or to potentially correct an R-trigger signal, the second data set may comprise ECG signals with stimulation (or pacing) and without stimulation (or pacing) of the heart and preferably enables different signal filtering of the ECG signals. Hence, it is advantageously possible to distinguish between patient's own ECG signals or physiologically or pathophysiologically induced ECG signals and stimulation-induced ECG signals, for example due to an electrical pulse of a pacemaker.

Preferably, the first data set comprises online ECG measurement signals of a currently performed extracorporeal circulatory support and the second data set comprises at least partially offline stored ECG signals. In other words, the first data set is received immediately after the ECG measurement signal is detected or recorded and preferably continuously, such that a quasi-simultaneous mapping is enabled. The second data set may be provided at least partially or completely offline. This allows the ECG signals in the second data set to be evaluated and analyzed before the first data set is received and, for example, the R-trigger validity in the second data set can be improved by predetermined data processing procedures. Thus, by combining offline analysis of data from previous circulatory supports and online analysis during a current circulatory support, the quality of the circulatory support can be documented and this comparison may then provide the framework for optimizing future (current) circulatory supports.

However, it may also be provided, alternatively or additionally, that initial data from the first data set is validated and annotated during the acquisition or recording of the first data set and extracorporeal circulatory support, wherein this data is used as part of the second data set or substantially form the second data set. In other words, the initially acquired and now annotated data of the (formerly) first data set may, through iteration(s), at least partially assume the role of the validated second data set. Thus, the evaluation or analysis of the data can also be staggered during the extracorporeal circulatory support to allow for a “near-line” analysis.

Accordingly, the device may be adapted to also store the first data set at least partially or completely, preferably for a predefined period of time. In this manner, the evaluation unit can take into account a course or progression of the measured ECG signal when mapping the second data set onto the first data set. This may increase the accuracy of the monitoring and, if necessary, the placement of the R-trigger signal.

A further possibility exists, wherein the R-R interval of the at least one preceding cardiac cycle is taken into account, such that the R-R intervals for successive cardiac cycles may be compared with each other. In other words, the signal may be output based on a comparison of the R-R interval for the present or actual cardiac cycle with the R-R interval for a preceding cardiac cycle, in particular the directly preceding cardiac cycle. For example, the time of the R-R interval of a carciac cycle may be positioned on an x-axis and plotted against the time of the R-R interval of a directly subsequent cardiac cycle on a y-axis, preferably in a Poincare diagram. Thereby, the course or a tendency of potential deviations for subsequent or successive cardiac cycles may be detected, for example based on the corresponding data points of the previous three cardiac cycles (R-Rₙ₋₂, R-Rₙ₋₁, R-Rn). On the other hand, this enables the definition of a confidence limit in order to unambiguously identify significant deviations.

Preferably, the data points are depicted for a predefined number of preceding cardiac cycles. The confidence limit may be mapped to the first data set and be based on the second data set, such that empirical values and validated data that are stored offline may be taken into account for the first data set or for the development of the R-R interval. A second data set may, alternatively or in addition, also be present as a reference value for the current first data set. Typically, the second data set does not include at least the current R-R interval.

Furthermore, a prediction of the R-R interval for a successive or subsequent future cardiac cycle (R-Rₙ₊₁) may occur based on such comparison. For example, a tendency may be determined based on the previous reference value and the current reference value of the R-R intervals. Also, it may be determined based on the empirical values from a second data set with an increased probability whether a subsequent or successive R-R interval may be outside of a confidence limit.

Accordingly, an advantageous monitoring of the R-R interval may be provided. A user may e.g. perform an adjustment of settings and/or may re-examine a patient's condition, if the confidence limit is (repeatedly) exceeded or in case data points accumulate at the confidence limit.

The signal to be output is furthermore indicative of whether an R-R interval of the first data set corresponds to an R-R interval corresponding to the respective cardiac cycles of the second data set that is mapped thereon. Accordingly, the signal to be output may be a warning signal or an R-trigger adapted to the R-triggers of the second data set, when the temporal distance exceeds a predetermined or predefined threshold. For example, the signal may comprise an acoustic warning signal or a visual marker or warning on a display, for example in a section for a timeline of the output trigger signals. Similarly, when the threshold is exceeded, a corrected R-trigger signal based on the corresponding ECG signal of the second data set may be output to correct an incorrectly placed R-trigger of the first data set and represent an optimized synchronization with an extracorporeal circulatory support.

Furthermore, the evaluation unit may be adapted to determine the temporal distance by means of correlation analysis, wherein the device is preferably adapted to output the signal, if a temporal distance of an R-trigger is outside a predefined confidence interval. R-R intervals of the first data set and the second data set may thus be correlated to each other. R-R intervals of the first data set that lie outside the confidence interval may indicate a faulty R-trigger and an existing trigger instability and may thus form the basis of the signal to be output.

In order to improve the accuracy of the measured ECG signal and to facilitate the mapping of the second data set, the evaluation unit may be adapted to evaluate the measured ECG signal temporally and/or spatially and to enable the mapping onto the evaluated ECG signal of the first data set.

Another possibility to increase the accuracy of the trigger stability is to use (i) left atrial and left ventricular transesophageal ECG signals and/or (ii) intercardiac atrial and ventricular ECG signals, with and without spatial and/or temporal signal averaging technique. The signal averaging technique may be implemented as sliding and/or non-sliding ECG averaging.

Thereby, the measured ECG signal of the first data set may comprise at least a first measurement signal from a first ECG lead and a second measurement signal from a second ECG lead, wherein the first and second ECG leads are spatially separated from each other and wherein the evaluation unit is preferably adapted to map the second data set onto the respective measurement signal or a spatially evaluated common measurement signal. The mapping of the second data set onto the first data set may be performed, for example, specifically to a particular measurement signal or a specific ECG lead or also to a measurement signal that is formed from multiple measurement signals.

For the temporal and/or spatial evaluation, different cardiac cycles or cardiac actions can be recorded in the predefined time period, wherein each cardiac cycle can define specified time points, for example, from a beginning of the cardiac cycle to the end of the cardiac cycle. This facilitates the comparison between different cardiac cycles, for example, compared to an evaluation using absolute time points. In this manner, the different cardiac cycle phases of successive cardiac cycles and, in particular, the course of these cardiac cycle phases of successive cardiac cycles may be compared with each other. Thus, data points are collected at-in successive cardiac cycles respective-identical time points. The collection of data points at identical times in the successive cardiac cycles is provided such that they can be compared with or offset to each other. Hence, a desired or useful signal can be displayed for each time point of a cardiac cycle phase.

Furthermore, for the same time point of a single cardiac cycle, different ECG leads may be provided to provide the ECG signal, such that a corresponding number of data points can be provided for each time point. The different measurement signals allow selective data points from specific ECG leads to be used for the processing.

Accordingly, in this preferred embodiment, at least two data points are available for each time point within the predefined or specified time period. However, depending on the number of cardiac cycles acquired and/or ECG leads present, more data points may be provided for each time point. The predefined time period may be defined, for example, by the treatment duration or also by the predefined number of acquired or detected cardiac cycles.

The spatial and/or temporal evaluation thus enables a correction of individual interfering signals, such that the determination of the at least one amplitude change within the cardiac cycle is facilitated and the accuracy is improved.

A spatial evaluation is based on the fact that the spatial separation of the leads and the corresponding signals, wherein, for example, there may be a spatial and/or anatomical spacing, may on the one hand ensure that the distance of the desired or useful signal from certain interfering signals, for example, from a stimulation of the heart, is improved. These interfering signals can thus be avoided to the greatest possible extent and at least partially filtered out such that they do not interfere with the detection of an R-wave or the generation of an R-trigger. On the other hand, this enables that an ECG signal with the strongest possible useful signal to be acquired even in the event of variations or variations in physiological signals, for example, of the native excitation leads, due to the spatial separation of the ECG leads.

The corresponding signals may be improved by “signal averaging”, wherein the evaluation unit is adapted to perform an addition or summation or averaging of the measurement signals or the respective, spatially separated data points. The ratio of the useful signal to the interference signal can be increased by a factor of at least 1.2, for example 1.4, such that the provision of an R-trigger can also take place in the case of weaker measurement signals or fluctuations. This may also provide an optimized trigger stability and more accurate monitoring.

Hence, the ratio of the useful signal to the interference signal may be improved for a number of n ECG leads by a factor of the square root of n. Accordingly, for two ECG leads, an improvement of √(n=2)=1.41 may be achieved. This improvement may be achieved, for example, in the presence of ideal noise with all frequencies. However, such improvement may be reduced in the presence of non-ideal noise signals, which may occur, for example, in the presence of biosignal interference.

A corresponding improvement in the ratio of the useful signal to the noise or interference signal can also be achieved with a temporal averaging or “signal averaging”. The improvement results from the square root number n of averaged cardiac actions or cardiac cycles, for example, from at least two averaged R-wave triggered cardiac actions.

The temporal averaging, i.e., the establishment of an average, or addition of the data points thus allow individual outliers, which, for example, do not lie in a relevant cardiac cycle range and are thus not characteristic of a particular cardiac cycle phase, not to impair the provision of an R-trigger, since the magnitude of the corresponding data point for other cardiac cycles is relatively small.

Although the averaging or addition with data points from only two cardiac cycles will improve the useful signal over the interfering signal, averaging or addition over more than two cardiac cycles, preferably as a sliding average, is preferably provided, e.g., over at least 10 or 10 to 100 cardiac cycles, preferably at least 40, e.g., between 40 and 80 cardiac cycles. As described above, a (theoretical) improvement of the useful signal can be increased by a factor corresponding to the square root, i.e. V n, for a number of n cardiac cycles or cardiac actions. If 25 cardiac cycles are averaged, the useful signal or the distance to an interference signal may be improved by a (theoretical) factor of 5.

The number of cardiac cycles is not fixed to an upper limit. Accordingly, more than 100 cardiac cycles may be detected or recorded, for example to compensate for relatively prominent outliers. However, data points of only 10 to 40 cardiac cycles can also be evaluated, for example, to enable a highly reactive adaptation to a changed physiological state.

Also for the temporal evaluation of the data points, the ECG signal may comprise at least a first measurement signal from a first ECG lead and a second measurement signal from a second ECG lead. In this regard, the data points from the two measurement signals may form a value together, for example, such that the data points are averaged both temporally and spatially. For example, at least one of the ECG leads may be formed as a transesophageal ECG lead and a corresponding probe. This approach has the advantage that the distance to a possible interfering signal, for example, in case of stimulation of the heart, and thus the useful signal can be accordingly improved.

Furthermore, a temporal averaging and spatial addition of the data points may also be provided. In this case, for example, data points from at least two measurement signals from spatially separated ECG leads can be added for the respective time point, and the added data points can then be averaged or an average may be formed for two or more cardiac cycles. In this manner, the ratio between the useful signal and the interfering signal as well as the stability of a trigger signal are further improved.

Preferably, the evaluation unit is furthermore adapted to multiply the respective data points or the evaluated data points, in particular to exponentiate them, preferably with a factor or exponent of greater than 1.3. Particularly preferably, the factor or exponent is 1.3 to 5.0 or 1.3 to 3.0 or 1.3 to 2.0. In this manner, the data points or the individual measurement signals are further improved, wherein higher measurement values are more prominent compared to lower measurement values due to the exponentiation and a potential interference signal can be reduced. The factor or exponent may depend on both a detection or recording frequency and a number of detected and evaluated cardiac cycles.

The above object is also solved by a control and regulation unit for an extracorporeal circulatory support. The control and regulation unit comprises a device described above for monitoring a temporal trigger stability of an extracorporeal circulatory support. The control and regulation unit is adapted to output a control and/or regulation signal for the extracorporeal circulatory support at a predefined time point after a respective R-trigger and taking into account the signal to be output.

In the control and regulation unit, the device may preferably be provided as an integrated ECG device or be attached to the control and regulation unit as an ECG device. Thus, the control and regulation unit can be used independently of the presence of other components and may be compact in design. Preferably, the ECG device is integrated into a single housing of a system for extracorporeal circulatory support, for example in a sensor box in the form of an ECG card or an ECG module.

The control and regulation unit may furthermore be housed in a console having a user interface for entering and reading settings of the system, in particular parameters of the blood pump of an extracorporeal circulatory support system and/or the ECG device. For example, the console may comprise a touchscreen and/or a display with a keyboard, which may be operated by a user. The control and regulation unit operates, actuates, controls, regulates and monitors the blood pump and enables synchronization of the blood pump with the cardiac cycle and, in particular, the provided R-trigger of the respective patient.

The above object is solved by a method for monitoring and optimizing a temporal trigger stability of an extracorporeal circulatory support. The method comprises at least the following steps:
receiving a first data set of a measurement of an ECG signal of a supported patient over a predefined time period;
determining or identifying a plurality of R-triggers from the first data set;
receiving or providing a second data set comprising evaluated ECG signals and a plurality of R-triggers;
selectively mapping the second data set onto the first data set; and
outputting a signal indicative of a temporal distance of successive R-triggers from the first data set and successive R-triggers from the second data set mapped thereon.

Advantageously, the second data set may comprise a plurality of validated clinical ECG signal measurements and/or simulated ECG signal data.

Preferably, the second data set comprises ECG signals with stimulation and without stimulation of the heart.

Preferably, the first data set comprises on-line ECG measurement signals from a currently performed extracorporeal circulatory support, wherein the second data set preferably comprises ECG signals stored at least partially off-line.

As described above, the signal to be output is furthermore indicative of whether an R-R interval of the first data set corresponds to an R-R interval corresponding to the respective cardiac cycles of the second data set being mapped thereon. Accordingly, if the temporal distance exceeds a predefined threshold, a warning signal or an R-trigger adapted to the R-triggers of the second data set can be output as a signal.

The temporal distance may furthermore be determined by correlation analysis for improved monitoring, wherein the signal is output when a temporal distance of an R-trigger falls outside a predefined confidence interval.

The selective mapping advantageously includes temporal and spectral mapping.

Furthermore, the accuracy of the measured ECG signal may be improved and the mapping of the second data set may be facilitated by evaluating the measured ECG signal temporally and/or spatially and basing the mapping on the evaluated ECG signal of the first data set. The measured ECG signal of the first data set may preferably comprise at least a first measurement signal from a first ECG lead and a second measurement signal from a second ECG lead, wherein the first and second ECG leads are spatially separated from each other and wherein the second data set is mapped on the respective measurement signal or a spatially evaluated common measurement signal.

Further advantages as well as possible embodiments and further embodiments of the methods have already been described in detail with respect to the control and regulation unit described above, such that a repeated description of the corresponding aspects is omitted in order to avoid redundancies. However, the corresponding disclosures equally apply to this subject matter.

### BRIEF DESCRIPTION OF THE DRAWINGS

Preferred further embodiments of the invention are explained in more detail in the following description of the Figures.

FIG. 1 shows a schematic representation of a device for monitoring a temporal trigger stability;

FIG. 2 shows an electrocardiographic of spatially separated ECG leads and an R-wave marker of the ECG signal;

FIG. 3 shows the electrocardiographic for the respective ECG leads according to FIG. 2 with annotated R-waves;

FIG. 4 shows the electrocardiographic course according to FIG. 2 with the annotated R-waves according to FIG. 3;

FIG. 5 shows an automated QRS detection of an ECG signal;

FIG. 6 shows a simulated comparison of annotated R-waves and automatically detected R-waves by mapping a second data set onto a first data set;

FIG. 7 shows a comparison of annotated R-waves and automatically detected R-waves by mapping a second data set onto a first data set with clinical data;

FIG. 8 shows a correlation analysis in a scattering matrix for R-R intervals of a first data set and R-R intervals of a second data set;

FIG. 9 shows a short-term Fourier transformation of R-R intervals for different heart rhythms; and

FIG. 10 shows an example of reference values for R-R intervals between successive cardiac cycles in a Poincaré diagram.

### DETAILED DESCRIPTION OF PREFERRED EMBODIMENTS

In the following, preferred embodiments will be explained in more detail with reference to the accompanying Figures. In the Figures, corresponding, similar, or like elements are denoted by identical reference numerals and repeated description thereof may be omitted in order to avoid redundancies.

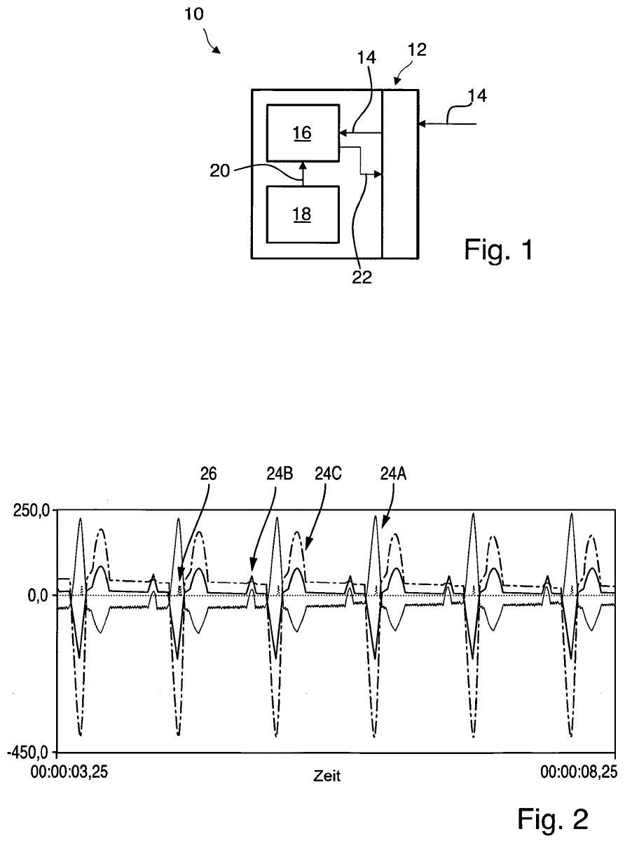

A schematic representation of a device 10 for monitoring a temporal trigger stability according to the present invention is shown in FIG. 1. In this embodiment, the device 10 is formed as an ECG module. Via an interface 12, data sets with measurement signals of an ECG signal can be received and signals can be transmitted to further devices or appliances without requiring any particular coupling. For example, the interface may enable communicative coupling with an extracorporeal circulatory support system or device, or may be formed together with an interface of a corresponding control and regulation unit.

Optionally, the device 10 can be formed as an ECG device that can be integrated into or attached to a control and regulation unit such that a control and regulation unit of an extracorporeal circulatory support device may be provided, which may be used independently of the provision of other components and is compactly formed. Preferably, the device 10 may be integrated into a single housing of an extracorporeal circulatory support system, such as a sensor box.

A first set of data 14 comprising measurement signals of an ECG signal of a supported patient may be received via the interface 12. The corresponding data may be temporarily stored in a memory and optionally stored in a storage 18 of the device. The first data set 14 is forwarded from the interface 12 or via the memory to an evaluation unit 16. The evaluation unit 16 is furthermore communicatively connected to the storage 18 and is adapted to receive a second data set 20 comprising evaluated ECG signals and to selectively map the second data set 20 onto the first data set 14. In the present example, the evaluation unit 16 is adapted to determine a plurality of R-waves or R-triggers from the first data set 14 that may serve to control and synchronize a blood pump of an extracorporeal circulatory support system. Furthermore, the second data set 20 also includes a plurality of R-triggers.

In the present embodiment, the evaluation unit 16 is further adapted to determine a temporal distance of successive R-triggers from the first data set and successive R-triggers from the second data set being mapped thereon. In other words, an R-R interval from the first data set 14 determined by the evaluation unit 16 is compared to a corresponding R-R interval from the second data set 20. This temporal distance serves as a measure of a signal 22 to be output by the device 10. In the present embodiment, the signal 22 is provided at the interface 12 and can be retrieved or received, for example, by a control and regulation unit.

The signal 22 can be output as a warning signal or also as an R-trigger adapted to the R-triggers of the second data set 20, if the temporal distance exceeds a predefined threshold value, which may be provided to the evaluation unit 16 by means of the storage 18.

Exemplary measurement signals of a data set, which can be annotated and validated and thus form the basis for a second data set to be provided, are shown in FIG. 2. An electrocardiographic course of spatially separated ECG leads is shown. These are simulated data from a patient receiving circulatory support with different bradycardic and tachycardic arrhythmias and an implanted dual-chamber pacemaker in DDD mode.

An ECG signal with a first 24A, second 24B, and third 24C measurement signal is shown. The corresponding ECG leads can be of transthoracic or transesophageal nature and thus detect and provide different measurement signals in the same cardiac cycle. Based on the ECG signal, further R-waves 26 had been identified using an ECG card and corresponding R-triggers had been provided, which serve to control an extracorporeal circulatory support.

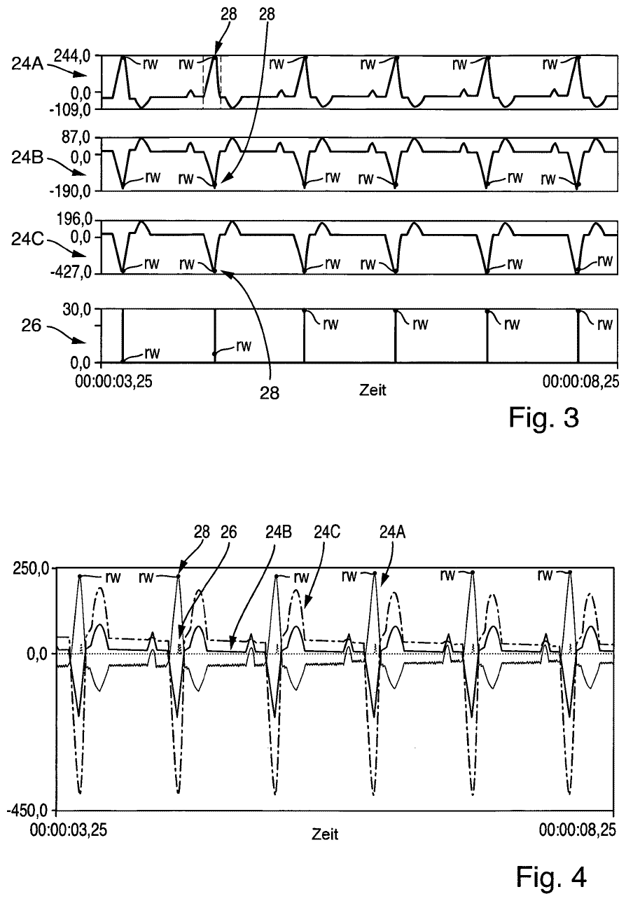

In FIG. 3, the measurement signals 24A-C from FIG. 2 are shown separately from each other, wherein the R-waves of the respective measurement signals 24A-C have been annotated as “rw” or R-waves 28. It can furthermore be seen where the annotated R-trigger 28 is located within a QRS complex or how it behaves, as indicated by the dashed lines.

In FIG. 4, the annotated R-waves 28 shown in FIG. 3 have been recorded in the course as shown in FIG. 2. It can be seen that the time points of the annotated R-waves 28 substantially correspond to the time points of the R-waves 26 identified or detected by the ECG card.

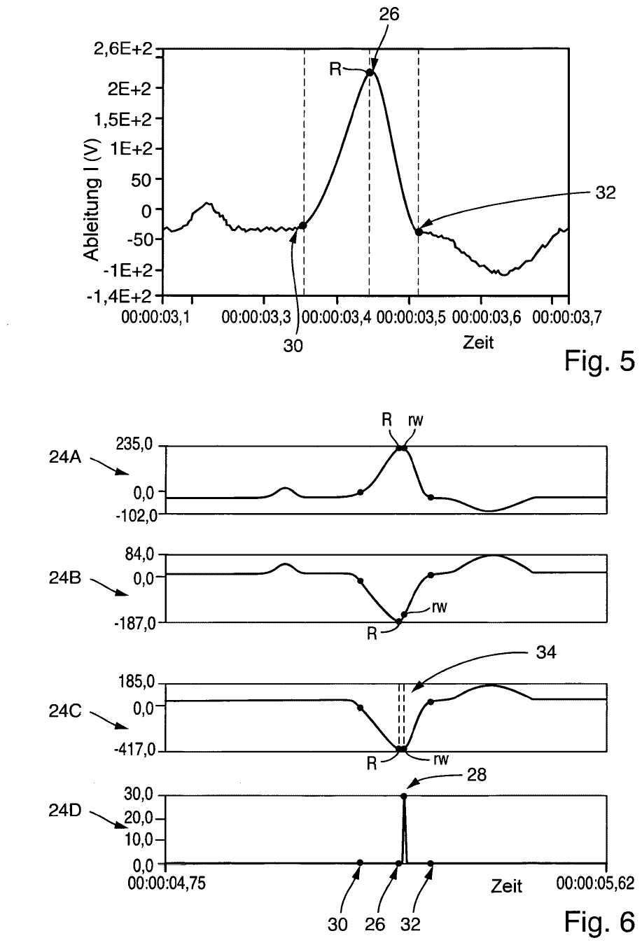

A general automated QRS detection of an ECG signal is shown in FIG. 5 for a single cardiac action in a simulated data set. In this case, an automatically detected R-wave with the R-wave marker 26 (“R”) as well as the QRS start 30 and the QRS end 32 were determined using a preset feature extraction. The feature extraction can be adapted on the basis of various parameters, for example by specifying an expected maximum heart rate, an expected frequency range of the QRS complex, an expected duration of the QRS onset 30 and/or an expected duration of the QRS end 32.

In FIG. 6, accordingly annotated R-waves 28 from a second data set have been mapped onto a first data set with different measurement signals 24A-C, analogous to FIG. 3, wherein a determined temporal difference 34 between the automatically acquired R-wave 26 (“R”) and the annotated R-wave 28 (“rw”) is shown in the present example, as indicated by the inserted lines. For clarity, the labeling of the various features has been shown only for the measurement signal 24C and the R-wave signal 26.

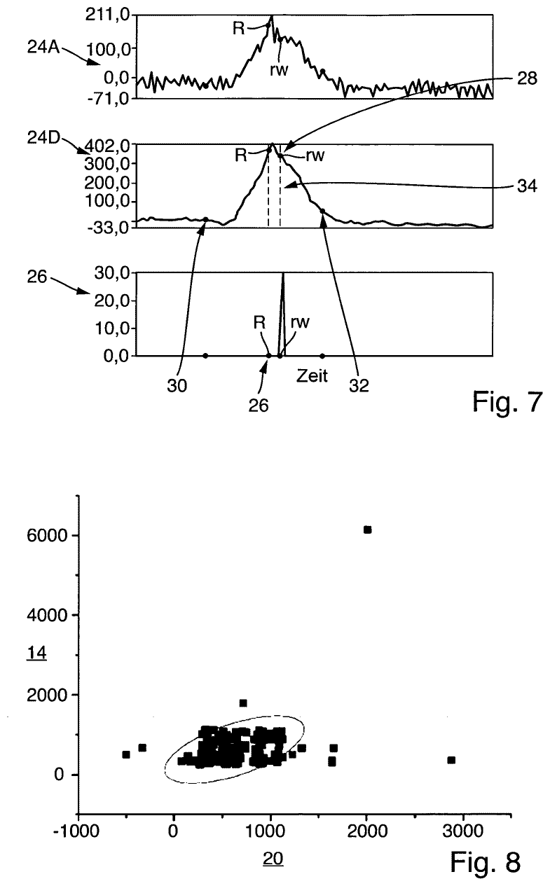

The data shown in FIG. 6 is initially simulated data. A mapping of a second data set onto measurement signals of a current first data set is furthermore shown in FIG. 7, wherein the ECG measurement signals correspond to clinical data. The data have been processed first, for example, using bandpass filters and/or signal averaging or signal averaging (24D). Again, it can be seen that a temporal difference between the automatically acquired R-wave 26 (“R”) and the annotated R-wave 28 (“rw”) can be successfully determined or detected.

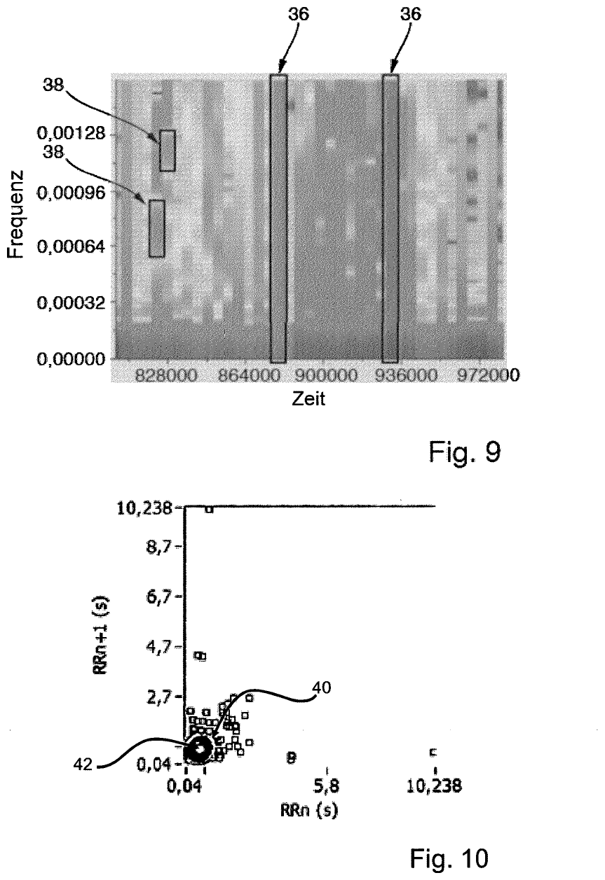

Based on the automatically detected R-waves 26 and the annotated R-waves 28, corresponding R-R intervals may be determined. An example of such resulting R-R intervals according to the first data set 14 and the second data set 20 is shown in FIG. 8 as a scattering matrix for R-R intervals against each other to enable a correlation analysis. The corresponding data are shown, wherein a condition can be specified on the basis of the (specified or variable) ellipsoid, for example, in order to assess the significance of a potential deviation. With the ellipsoid shown here, it is clarified that the available data are predominantly matching. Only single outliers can be observed (e.g. at r=0.6, P>0.001 and 95% confidence interval). Alternatively, or in addition to such a correlation matrix, a Fourier transformation of the determined R-R intervals can also be provided in order to specify (further) conditions for acceptable R-R correlations. FIG. 9 shows a corresponding short-term Fourier transformation of R-R intervals of a patient with a normofrequent, bradycardic and tachycardic heart rhythm.

Using the corresponding “heat map”, conditions can be established, for example, according to which the R-R correlation is essentially independent of frequency. This can be identified by having an essentially homogeneous distribution of frequency for a given time point (a consistently high temperature, so to speak, for each frequency at that time point). This is shown in FIG. 9 with the exemplary rectangles which have been marked with the reference sign 36. Alternatively, for example, a condition can be defined which is frequency and time dependent, which is shown in FIG. 9 by the different temperatures or the inhomogeneous distribution for a certain time point. This is shown in FIG. 9 with the exemplary rectangles, which have been marked with the reference sign 38. In this manner, different conditions can be provided based on the analyzed and annotated data to optimize the trigger instability.

In FIG. 10 reference values of R-R intervals between two successive cardiac cycles are depicted in a Poincaré diagram. In said diagram data points are depicted for a predefined number of preceding cardiac cycles, wherein the data points are continuously refreshed for a predefined, present time. As shown, the R-R intervals (RR) are more dense in a lower value region for the preceding (RRn) and the present (RRn+1) cardiac cycle. It follows that the R-R intervals for successive cardiac cycles generally only show minor deviations.

Furthermore, a circular confidence limit 40 is depicted, which preferably is based on validated data from an offline stored second data set. Alternatively, the confidence limit 40 may also be automatically selected based on a distribution of the data points. For example, a percentage of the currently included data points may be predefined and/or a maximum deviation to a determined statistical value may be taken into account.

In the diagram the present R-R interval or the present reference value 42 is depicted as a bright (er) point, which may be unambiguously perceived by a user. The R-R interval according to this example lies exactly in the middle of the confidence limit 40. As explained above, a course or tendency of potential deviations may be determined for subsequent cardiac cycles, which may accordingly be depicted. For example, the previous three reference values may be depicted or plotted with different colors and/or brightness. Accordingly, the attention of the user may be drawn as soon as possible to failure potentially occurring presently without requiring a manual intervention to identify such discrepancies or inconsistencies.

Based on the diagram, a value range may furthermore be depicted for the directly subsequent or successive future cardiac cycle. The value range may e.g. be determined based on a determined tendency of the present reference values 42 and on offline stored and validated reference values, which may be mapped to the present reference values.

Where applicable, all the individual features depicted in the exemplary embodiments may be combined and/or exchanged without leaving the scope of the invention.

### LIST OF REFERENCE NUMERALS

10 Device
12 Interface
14 First data set
16 Evaluation unit
18 Storage
20 Second data set
22 Signal
24A First measurement signal
24B Second measurement signal
24C Third measurement signal
24D Processed data
26 R-wave mark or automatically detected R-wave (“R”)
28 R-wave annotation (“rw”)
30 QRS start
32 QRS end
34 Difference
36 Frequency-independent condition
38 Frequency- and time-dependent condition
40 Confidence limit
42 Present reference value

1. A device (10) for monitoring and optimizing a temporal trigger stability of an extracorporeal circulatory support, the device being adapted to
receive a first data set (14) of a measurement of an ECG signal of a supported patient over a predefined period of time,
wherein the device (10) comprises an evaluation unit (16) adapted to determine or identify a plurality of R-triggers (26) from the first data set (14), the evaluation unit (16) being furthermore adapted to receive or provide a second data set (20) comprising evaluated ECG signals and a plurality of R-triggers (28) and to selectively map the second data set (20) onto the first data set (14),
wherein the device is adapted to output a signal (22) indicative of a temporal distance of successive R-triggers (26) from the first data set (14) and successive R-triggers (28) from the second data set (20) being mapped thereon.

2. The device (10) according to claim 1, wherein the second data set (20) comprises a plurality of validated clinical ECG signal measurements and/or simulated ECG signal data.

3. The device (10) according to claim 2, wherein the second data set (20) comprises ECG signals with stimulation and without stimulation of the heart.

4. The device (10) according to any of the preceding claims, wherein the first data set (14) comprises online ECG measurement signals of a currently performed extracorporeal circulatory support and the second data set (20) comprises at least partially offline stored ECG signals.

5. The device (10) according to any of the preceding claims, wherein the device is configured to output the signal based on a comparison of the R-R interval of the present cardiac cycle with an R-R interval of a preceding, in particular the directly preceding cardiac cycle.

6. The device (10) according to any of the preceding claims, wherein the signal (22) to be output is a warning signal or an R-trigger adapted to the R-triggers (28) of the second data set (20), if the temporal distance exceeds a predefined threshold.

7. The device (10) according to any of the preceding claims, wherein the evaluation unit (16) is adapted to determine the temporal distance by means of correlation analysis, wherein the device (10) is adapted to output the signal (22), if a temporal distance of an R-trigger (26) lies outside a predefined confidence interval.

8. The device (10) according to any of the preceding claims, wherein the selective mapping comprises temporal and spectral mapping.

9. The device (10) according to any of the preceding claims, wherein the evaluation unit (16) is adapted to evaluate the measured ECG signal temporally and/or spatially and to enable the mapping on the evaluated ECG signal of the first data set (14).

10. The device (10) according to any of the preceding claims, wherein the measured ECG signal of the first data set (14) comprises at least a first measurement signal (24A) from a first ECG lead and a second measurement signal (24B, 24C) from a second ECG lead, wherein the first and second ECG leads are spatially separated from each other, and wherein the evaluation unit (16) is adapted to map the second data set (20) onto the respective measurement signal (24A-24C) or a spatially evaluated common measurement signal.

11. A control and regulation unit for an extracorporeal circulatory support, comprising a device according to any of the preceding claims, wherein the control and regulation unit is adapted to output a control and/or regulation signal for the extracorporeal circulatory support at a predefined time point after a respective R-trigger and taking into account the signal to be output.

12. A method for monitoring and optimizing a temporal trigger stability of an extracorporeal circulatory support, comprising the steps of:
receiving a first data set of a measurement of an ECG signal from a supported patient over a predefined time period;
determining or identifying a plurality of R-triggers from the first data set;
receiving or providing a second data set comprising evaluated ECG signals and a plurality of R-triggers;
selectively mapping the second data set onto the first data set; and
outputting a signal indicative of a temporal distance of successive R-triggers from the first data set and successive R-triggers from the second data set being mapped thereon.

13. The method of claim 12, wherein the second data set comprises a plurality of validated clinical ECG signal measurements and/or simulated ECG signal data.

14. The method according to claim 13, wherein the second data set comprises ECG signals with stimulation and without stimulation of the heart.

15. The method according to any of claims 12 to 14, wherein the first data set comprises online ECG measurement signals of a currently performed extracorporeal circulatory support and the second data set comprises at least partially offline stored ECG signals.

16. The method according to any of claims 12 to 15, wherein the signal is output based on a comparison of the R-R interval of a present cardiac cycle with the R-R interval of a preceding cardiac cycle, in particular of the directly preceding cardiac cycle.

17. The method according to any of claims 12 to 16, wherein, when the temporal distance exceeds a predefined threshold, a warning signal or an R-trigger adapted to the R-triggers of the second data set is output as a signal.

18. The method according to any of claims 12 to 17, wherein the temporal distance is determined by means of correlation analysis and wherein the signal is output, if a temporal distance of an R-trigger lies outside a predefined confidence interval.

19. The method according to any of claims 12 to 18, wherein the selective mapping comprises temporal and spectral mapping.

20. The method according to any of claims 12 to 19, wherein the measured ECG signal is evaluated temporally and/or spatially and the mapping is based on the evaluated ECG signal of the first data set.

21. The method according to any of claims 12 to 20, wherein the measured ECG signal of the first data set comprises at least a first measurement signal from a first ECG lead and a second measurement signal from a second ECG lead, wherein the first and second ECG leads are spatially separated from each other, and wherein the second data set is mapped onto the respective measurement signal or a spatially evaluated common measurement signal.
